(19) United States
(12) Patent Application Publication
(10) Pub. No.: US 2023/0190352 A1
(43) Pub. Date: Jun. 22, 2023

(54) BONE ALIGN AND JOINT PREPARATION DEVICE AND METHOD

(71) Applicant: MEDARTIS AG, Basel (CH)

(72) Inventor: Matthew COYNE, Warsaw, IN (US)

(73) Assignee: MEDARTIS AG, Basel (CH)

(21) Appl. No.: 18/145,586

(22) Filed: Dec. 22, 2022

Related U.S. Application Data
(60) Provisional application No. 63/265,873, filed on Dec. 22, 2021.

(51) Int. Cl. A61B 17/88; A61B 17/17

(52) U.S. Cl. CPC A61B 17/8866; A61B 17/8869; A61B 17/1775

(57) ABSTRACT

A bone displacement system includes an anchoring portion, a flexing mechanism, and a distal body. The anchoring portion has an aperture for receiving a wire to connect the anchoring portion to a proximal bone. The flexing mechanism is connected to the anchoring portion, and has a rail and a holding portion movable on the rail. The rail is curved about a center of rotation located toward a bottom of the flexing mechanism and toward the proximal bone. The distal body is connected to the holding portion. The distal body has an aperture for receiving a wire to connect the distal body to a distal bone. The flexing mechanism is configured to move the distal body relative to the anchoring portion by a movement of the holding portion on the rail and about the center of rotation to flex the first bone relative to the second bone.

### CROSS-REFERENCE TO RELATED APPLICATIONS

The present application claims priority to U.S. Provisional Application Ser. No. 63/265,873 filed on Dec. 22, 2021 (Attorney Docket No. 3768.121P1), which is incorporated herein by reference in its entirety.

The present application is related to U.S. application Ser. No. 17/238,920 (Attorney Docket No. 3768.099A) filed Apr. 23, 2021, which is related to U.S. application Ser. No. 17/022,761 (Attorney Docket No. 3768.090A) filed Sep. 16, 2020, which claims the benefit of U.S. Provisional Application Ser. No. 62/900,920 (Attorney Docket No. 3768.090P1) filed Sep. 16, 2019, U.S. Provisional Application No. 62/991,879 (Attorney Docket No. 3768.094P1) filed Mar. 19, 2020, and U.S. Provisional Application No. 63/015,052 (Attorney Docket No. 3768.099P1) filed Apr. 24, 2020, which are incorporated herein by reference in their entireties. The present application is related to U.S. Provisional Application Ser. No. 62/015,052 filed on Apr. 24, 2020, which is incorporated herein by reference in its entirety.

The present application is related to U.S. application Ser. No. 17/022,761 (Attorney Docket No. 3768.090A) filed Sep. 16, 2020, which claims the benefit of U.S. Provisional Application Ser. No. 62/900,920 (Attorney Docket No. 3768.090P1) filed Sep. 16, 2019, U.S. Provisional Application No. 62/991,879 (Attorney Docket No. 3768.094P1) filed Mar. 19, 2020, and U.S. Provisional Application No. 63/015,052 (Attorney Docket No. 3768.099P1) filed Apr. 24, 2020, which are incorporated herein by reference in their entireties.

### FIELD OF THE INVENTION

This application relates to apparatuses, devices, and methods for adjusting and joining bones.

### DESCRIPTION OF THE RELATED ART

Collapsing foot deformity is the medical term for flat foot which occurs when the arches on inside of a person's foot are flattened. This may cause pain in a heal or arch area. Flat foot may be of a flexible or rigid variety with the latter variety often caused by abnormal bone conditions in the foot.

Hallux valgus is the medical term for a bunion. The first tarsal-metatarsal (TMT) joint is an important joint at the inner part of the middle of the foot. The two bones that meet to form this joint are the first metatarsal and medial cuneiform bones. When this joint has too much looseness or movement, the condition is known as hypermobility or instability. When this joint becomes hypermobile, the first metatarsal moves too much in one direction and the first toe compensates by moving too much in the other direction. When this happens, a bunion develops.

The bunion is a disease of the joint and soft tissue. A bunion deformity or hallux abducto valgus deformity results from the big toe deviating laterally toward the patient's smallest toe. Due to the lateral movement of the big toe, the first metatarsal bone angles toward the smaller toes on the patient's foot causing the first metatarsal bone to move out of alignment. Bunions may become irritating and, in some cases, very painful during walking and other weight bearing activities. Bunions may also be painful and debilitating condition that prevents wearing shoes. Genetics and poor shoe design are the causes. The angle between the metatarsal of the second digit is a means to quantify the degree of deformity.

Painful bunions are corrected by surgical soft tissue management and surgical bone reforming. The first metatarsal is corrected by sectioning it with a saw and moving the head laterally. There are numerous cut locations from the proximal to distal regions, namely the chevron, Ludloff, Mau and proximal. The bones are shifted, and held in place with screws, staples or plates. Sometimes adjacent joints are fused to stabilize the reconstruction.

The Lapidus procedure is a type of fusion of the first TMT joint that decreases the movement of that joint and straightens out the first metatarsal and toe, so the Lapidus procedure treats bunions caused by first TMT joint hypermobility.

The goal of the Lapidus procedure is to surgically treat hallux valgus that is caused by first TMT joint hypermobility. An orthopedic foot and ankle surgeon realigns to a normal toe shape by placing the first metatarsal straight with the medial cuneiform bone and locking or fusing these two bones together. When the first TMT joint is fused, the first metatarsal will not move abnormally. This will allow the first toe to stay straight and prevent the bunion from coming back.

Thus, a need exists for devices, systems, and methods for treating foot deformities that are repeatable yet adaptable to particular clinical situations.

### SUMMARY

The present invention provides, in a first aspect, a bone displacement system including an anchoring portion, a flexing mechanism, and a distal body. The anchoring portion has an aperture for receiving a wire to connect the anchoring portion to a proximal bone. The flexing mechanism is connected to the anchoring portion, and has a rail and a holding portion movable on the rail. The rail is curved about a center of rotation located toward a bottom of the flexing mechanism and toward the proximal bone. The distal body is connected to the holding portion. The distal body has an aperture for receiving a wire to connect the distal body to a distal bone. The flexing mechanism is configured to move the distal body relative to the anchoring portion by a movement of the holding portion on the rail and about the center of rotation to flex the first bone relative to the second bone.

The present invention provides, in a second aspect, a method for use in bone displacement, including inserting a first wire through an anchoring portion of a bone displacement mechanism into a first bone. The anchoring portion is connected to a curved rail. A second wire is inserted through a distal body of the bone displacement mechanism distal to the anchoring portion into a second bone distal to the first bone. The distal body is connected to a holding portion. The holding portion is moved on the curved rail to move the distal body relative to the anchoring portion and about a center of rotation of the curved rail to flex the first bone relative to the second bone.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 is a side view of a bone displacement system engaged with a foot in accordance with the present invention;

FIG. 2 is a perspective view of the system of FIG. 1;

FIG. 3 is side view of the system of the system of FIG. 1 after a space has been created between two bones of the foot;

FIG. 4 is a perspective view of the e system of FIG. 3;

FIG. 5 is a perspective view of the system of FIG. 3 with a wedge received in the space;

FIG. 6 is a side view of the system of FIG. 5;

FIG. 7 a perspectiveview of the system of FIG. 4 further including a bone plate;

FIG. 8 is a perspective view of the system of FIG. 7 with a screw through the bone plate into the wedge;

FIG. 9 is a perspective view of the system of FIG. 8 further including screws thorugh the plate into the bones;

FIG. 10 is a perspective view of the system of FIG. 5 further including a screw thorugh one of the bones into the wedge;

FIG. 11 is a perspective view of another embodiment of a bone displacement system including a flexing mechanism, compression-distraction mechanism and curved frontal plane rail mechanism in accordance with the present invention;

FIG. 12 a perspective view of an embodiment of a bone system including a flexing mechanism, compression-distraction mechanism .a curved frontal plane rail mechanism and an intermetatarsal curved rail mechanism in accordance with the present invention;

FIG. 13 is a perspective view of another embodiment of a bone displacement and preparation displacement a system including a modular cartridge portion, a flexing mechanism, a compression-distraction mechanism, a curved frontal plane rail mechanism, an intermetatarsal curved rail mechanism, and a lateral adjusting mechanism in accordance with the present invention;

FIG. 14 depicts a side view of the system of FIG. 13;

FIG. 15 is a top perspective view of the system of FIG. 13;

FIG. 16 is a perspective view of another embodiement of a bone displacement having a a flexing mechanism and a linear compression-distraction mechanism in accordance with the present invention;

FIG. 17 is a perspective view of another embodiement of a bone displacement having a a flexing mechanism in accordance with the present invention; and

FIG. 18 is a side view of the system of FIG. 17.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

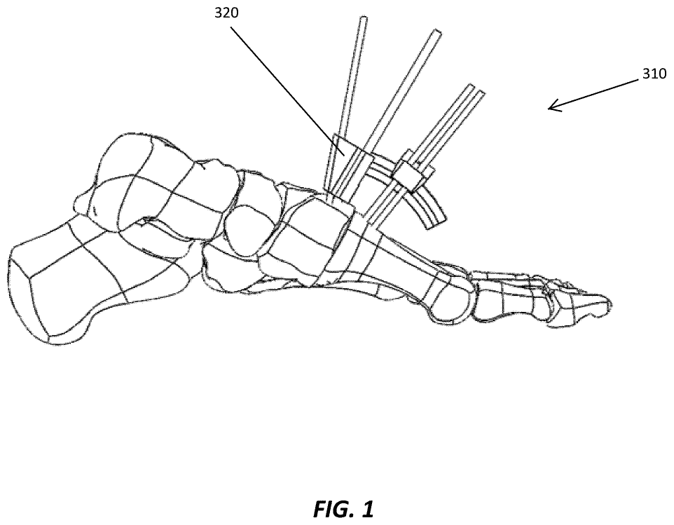

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

The following description references systems, methods, and apparatuses for cutting tools for orthopedic surgery involving a foot or lower extremities. However, those possessing an ordinary level of skill in the relevant art will appreciate that other extremities, joints, and parts of the musculoskeletal system are suitable for use with the foregoing systems, methods and apparatuses. Likewise, the various figures, steps, procedures and work-flows are presented only as an example and in no way limit the systems, methods or apparatuses described to performing their respective tasks or outcomes in different time-frames or orders. The teachings of the present invention may be applied to any orthopedic surgery, such as on the hand as well as other upper and lower extremities and may be implemented in other treatments sites that have similar anatomical considerations.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

As will be described below, the present invention includes systems and methods for correcting a deformity of the human foot. As depicted in FIGS. 1-10, a bone displacement system 310 may include a proximal anchoring portion 320, a connecting portion 330, a flexing mechanism 340 and a distal anchoring portion 350.

Proximal anchoring portion 320 may include a first anchoring hole 322 and a second anchoring hoe 324 therethrough for receiving a first K-wire 326 and a second K-wire 328, respectively for connecting anchoring portion 320 to a bone (e.g., a first medial cuneiform 115). For example, second k-wire 328 may be connected to a middle of first cuneiform 115, as depicted. Second anchoring hole 324 maybe aligned at an angle (e.g., at about 30-45 degrees) relative to first anchoring hole 322, as depicted. The alignment of the second K-wire at an angle (e.g., 20-45 degrees) relative to the first K-wire may inhibit a separation of anchoring portion 320 from first cuneiform 115, for example.

Proximal anchoring portion 320 may be connected to flexing mechanism 340 by connecting portion 330. Distal anchoring portion 350 may be connected to flexing mechanism 340. Distal anchoring portion 350 may include a third hole 356 and a fourth hole 384 therethrough for receiving a third K-wire 352 and a fourth K-wire 354, respectively. Anchoring portion 350 may be connected to a second bone (e.g., a first metatarsal 117) via third K-wire 356 and fourth K-wire 358.

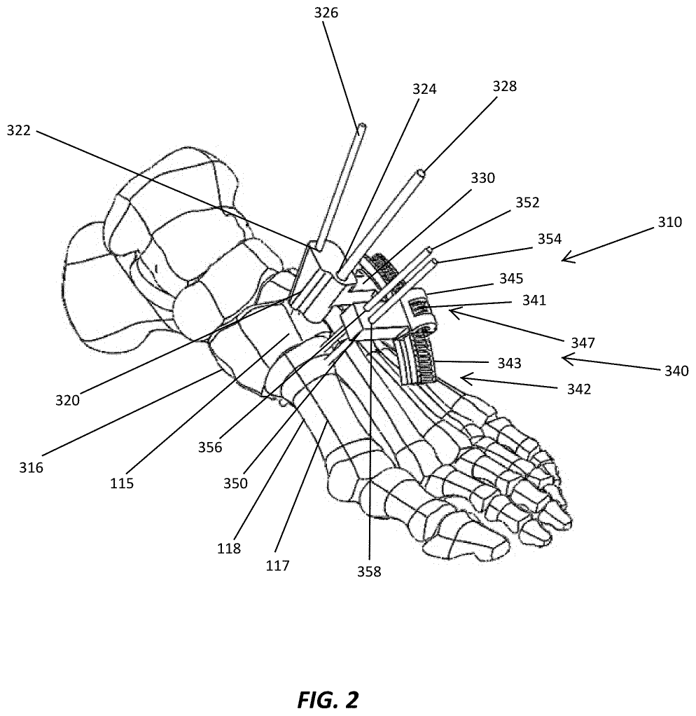

Flexing mechanism 340 may may include a mobile holding portion 345 holding a worm screw 341 received within a recess 347 and movably engaged with teeth 343 to move along an axis of a threaded rail 342. Rail 342 and axis may be curved such that a center of an arch forming the axis may be located toward the bones (e.g., first cuneiform 115 and first metatarsal 117) relative to a remainder of bone displacement system 310. For example, a center of rotation of holding portion 345 on rail 342 may be near a bottom face 316 of First medial cuneiform 115 and could range anywhere from a middle of the cuneiform to a bottom of a base 118 of first metatarsal 117, as depicted in FIG. 2.

As indicated above, distal anchoring portion 350 may be connected to flexing mechanism 340 and may be connected to a bone (e.g., first metatarsal 117) via one or more k-wires (e.g., third K-wire 356 and fourth K-wire 358) such that a movement of holding portion 345 on rail 342 may cause a movement of first medial cuneiform 115 connected to proximal anchoring portion 320 relative to first metatarsal connected to third K-wire 356 and/or fourth K-wire 358. Such movement of holding portion 345 on rail 342 may be caused by a rotation of worm screw 341 received within recess 347 by a user thereby causing worm screw to engage rail 342 to cause relative movement between rail 342 and holding portion 345.

In a example, collapsing foot deformity may be treated utilizing bone displacement system 310. As described above, proximal anchoring portion 320 may be connected to a bone (e.g., first cuneiform 115) by a K-wire (e.g., second k-wire 328) through a middle of the bone (e.g., first cuneiform 115), as depicted. Distal anchoring portion 350 may be connected to a bone (e.g., first metatarsal 117) via third K-wire 352 and fourth K-wire 354.

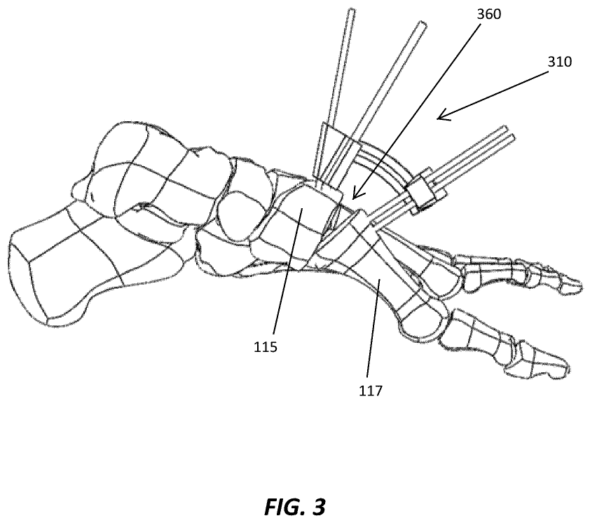

As described above, worm screw 341 may be moved along rail 342 to move holding portion 345 and thus third K-wire 352 and 4th K-wire 354 connected to first metatarsal 117 such that the first metatarsal 117 may rotate relative to first cuneiform about a center of an arch forming rail 342 as described above. This rotation results in plantar and/or dorsiflexion of first metatarsal 117 dependent on the direction the carriage is translated (e.g., along rail 342). At any point after the K-wires (e.g., third K-wire 352 and fourth K-wire 354) have been placed into the first metatarsal 117, the holding portion 345 may be rotated about a K-wire or pin (e.g., second K-wire 328) put into the center of the cuneiform (first cuneiform 115) to rotate the metatarsal (e.g., first metatarsal 11 about the pin (e.g., second K-wire 328), thus correcting the intermetatarsal angle of any potential bunion deformity as depicted for example, in FIG. 3. Further, a fourth pin or K-wire (e.g., first K-wire 326) may be inserted through a hole (e.g., first hole 322) into the medial cuneiform to lock the intermetatarsal angle (e.g., angle between axes of first cuneiform 115 and first metatarsal 117) after manipulation thereof by movement of holding portion 345 along rail 342.

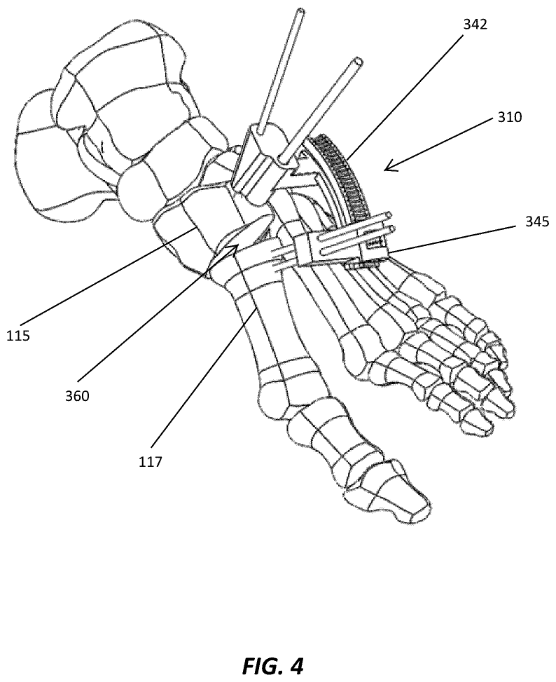

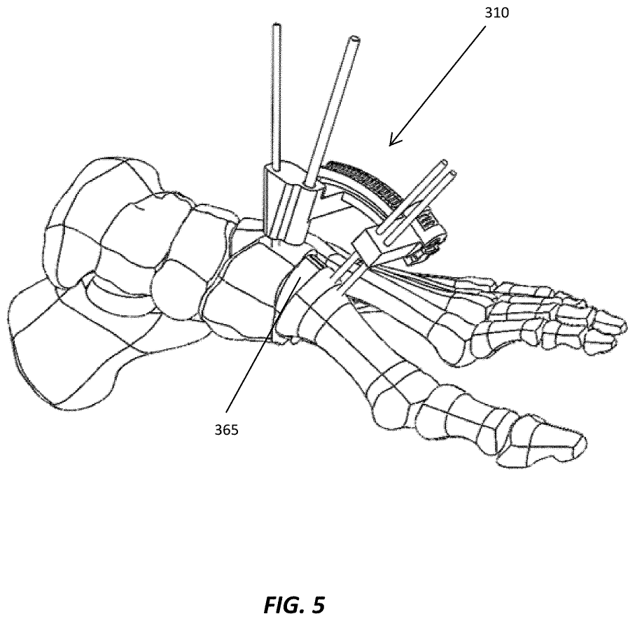

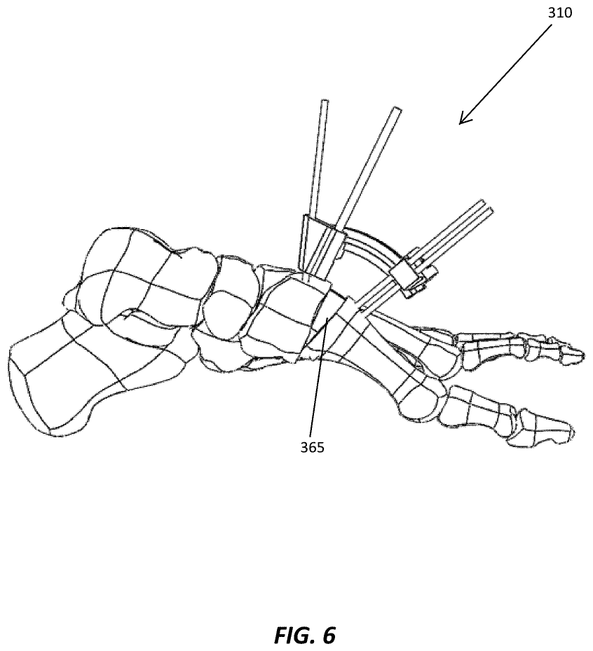

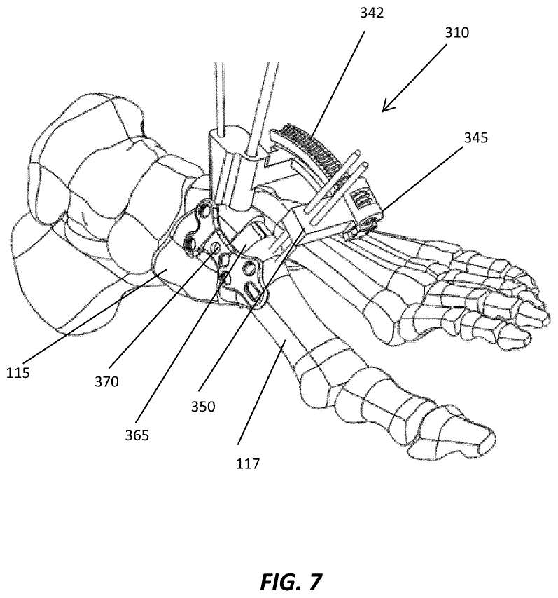

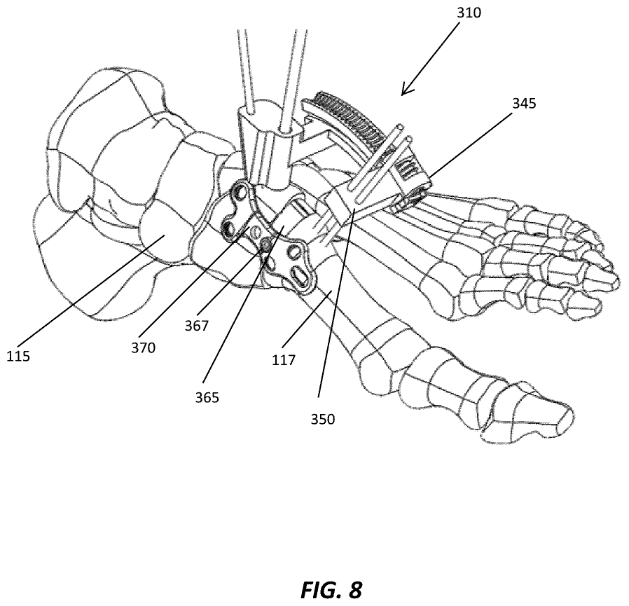

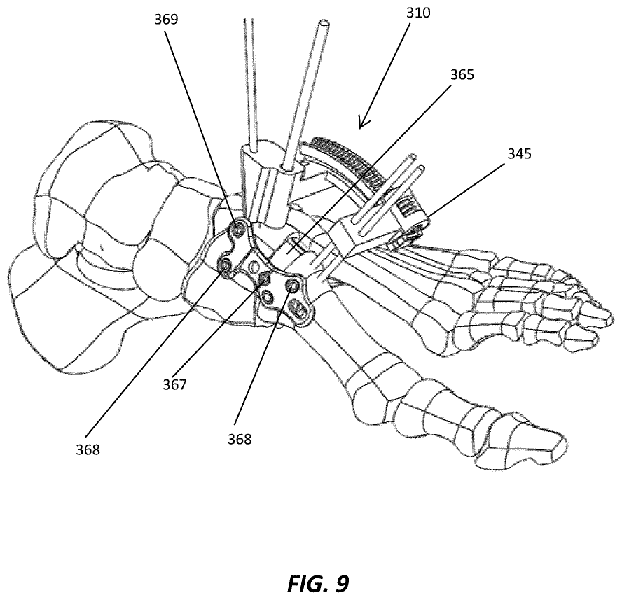

The correction of the metatarsal angle as described above may create a space or gap 360 between first cuneiform 115 and first metatarsal 117, as depicted in FIGS. 3-4. A wedge 365 made of bio friendly material (e.g., PEEK, stainless steel, bone graft) may be inserted in gap 360 created using bone displacement system 310 as described above to hold the bones (e.g., first cuneiform 115, first metatarsal 117) in a desired position, as depicted in FIGS. 5-6. A bone plate 370 may also be placed over the bones (e.g., first cuneiform 115, first metatarsal 117) as depicted in FIG. 7 and attached via screws, bolts or other fasteners to secure them relative to each other and to hold the wedge (i.e., wedge 365) in place as depicted in FIGS. 8-9. For example, a screw 367 of the screws through the plate (e.g., plate 370) may be threaded into in opening (or may be self starting to create an opening) of the wedge (e.g., wedge 365) to secure the wedge relative to the plate, as depicted in FIG. 8. Screws 368 may be threaded through remaining holes 369 of plate 370 into the bones (e.g., first cuneiform 115, first metatarsal 117) to attach the plate to the bones.

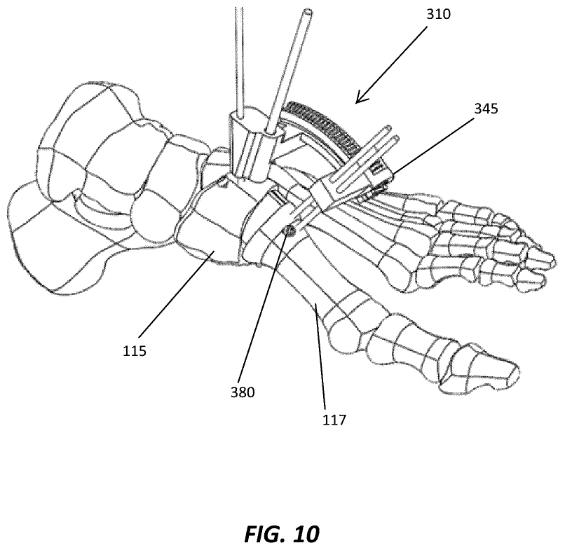

In another example, instead of attaching a plate to the bones and wedge, a screw 380 may be inserted through one of the bones (e.g., first metatarsal 117) into the wedge (e.g., wedge 365) to attach the wedge to one or more of the bones and maintain the wedge in a desired position, as depicted in FIG. 10. In a further example, such an additional screw may be attached through a bone (e.g., first metatarsal 117) into a wedge (e.g., wedge 365) as an addition to the described the attachment of a bone plate (e.g., bone plate 370).

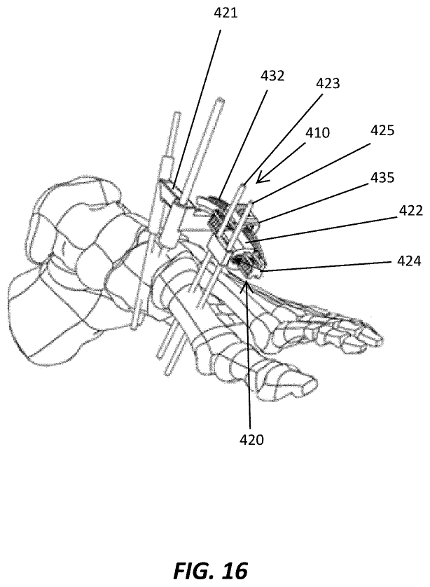

FIG. 16 depicts a bone displacement system 410, similar to bone displacement system 310, described above but with the addition of a linear compression/distraction mechanism 420, similar to compression distraction mechanism 40 disclosed in co-owned US patent application Ser. No. 17/238,920 (attorney docket number 3768.099A), wherein mechanism 420 is connected to a rail 432 connected to a holding portion 435 similar to rail 342 and holding portion 345 described above.

The utilization of mechanism 420 allows a treatment of wide range of deformities. As depicted in FIG. 16, bone displacement system 410 includes curved rail 432 connected to a proximal anchoring portion 421 and holding portion 435 engaged therewith, similar to bone displacement system 310 described above. Rail 342 may be connected to mechanism 420. A moveable holding portion 422 maybe engaged with a linear rail 424 with the movable holding portion attachable to a bone (e.g., first metatarsal 117) via a K-wire 423 and a K-wire 425 extendable through holes therein. Holding portion 422 may be translated along the linear rail (e.g., linear rail 424) which may translate the bone (e.g., first metatarsal 117) in distal and proximal directions resulting in compression/distraction of a cuneiform-metatarsal joint (e.g., a joint of medial cuneiform 115 and first metatarsal 117). Holding portion 435 may be translated along the curved rail (e.g., rail 432) thus rotating a first metatarsal (e.g., first metatarsal 117) about a center point of rotation as described above. This rotation may result in plantar and/or dorsiflex dependent on the direction the carriage is translated.

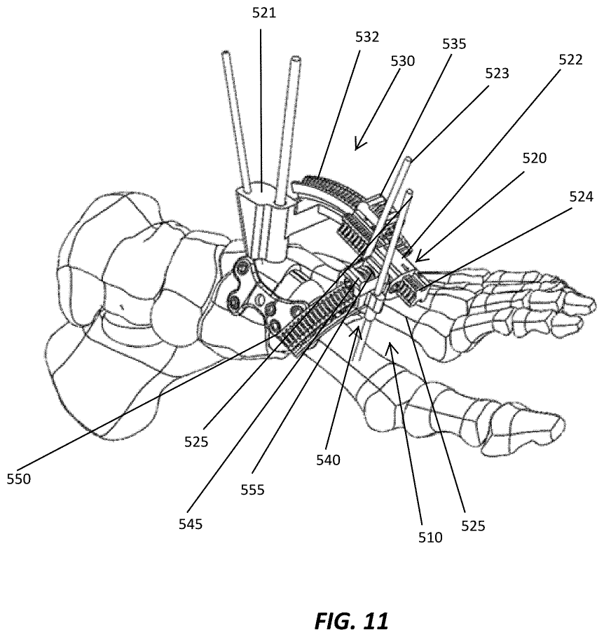

FIG. 11 depicts a bone displacement system 510 , similar to bone displacement system 310 , described above but with the addition of a linear compression distraction mechanism 520, similar to compression distraction mechanism 40 disclosed in co-owned US patent application Ser. No. 17/238,920 (attorney docket number 3768.099A), wherein mechanism 520 is connected to a flexing mechanism 530 having a rail 532 connected to a holding portion 535 similar to rail 342 and holding portion 345 described above. A curved frontal plane rail mechanism 540, similar to pronation alignment trolley 30 disclosed in the indicated patent application, is attached to mechanism 520.

The utilization of mechanism 520 and mechanism 540, in addition to flexing mechanism 530, allow a treatment of a wider range of bunion deformities than without these elements. As depicted in FIG. 11, flexing mechanism 530 includes curved rail 532 connected to a proximal anchoring portion 521 and holding portion 535 engaged therewith, similar to bone displacement system 310 described above. Rail 532 may be connected to mechanism 520. A moveable holding portion 522 may be engaged with a linear rail 524 with the movable holding portion attachable to a metatarsal (e.g., first metatarsal 117) via a K-wire 523 extendable therethrough. Holding portion 522 may be translated along the linear rail (e.g., linear rail 524) which may translate the first metatarsal (e.g., first metatarsal 117) in distal and proximal directions resulting in compression and/or distraction of a medial cuneiform- first metatarsal join (e.g., of first medial cuneiform 115 and first metatarsal 117). Holding portion 535 may be translated along the curved rail (e.g., rail 532) thus rotating the first metatarsal about a center point of rotation as described above. This rotation results in plantar and/or dorsiflex dependent on the direction the carriage is translated. Mechanism 540 may include a holding portion 545 engageable with a rail 550 such that the holding portion is movable along the rail via a worm gear day 555. Holding portion 545 may be connected to a bone (e.g., first metatarsal 117) via a K-wire 525. Rail 550 may be curved relative to the metatarsal such that a center of rotation of the curved rail falls within the first metatarsal similar to pronation alignment trolley 30 disclosed in indicated co-owned patent application Ser. No. 17/238,920 (Attorney docket number 3768.099A). Movement of holding portion 545 connected to rail 550 and the metatarsal (e.g., first metatarsal 117) may correct a frontal plane deformity of the first metatarsal. As indicated above, movement of holding portion 522 along rail 524 may translate the first metatarsal distally and proximally resulting in compression distraction of the medial cuneiform-first metatarsal joint as described above. Movement of holding portion 535 connected to rail 532 of flexing mechanism 530 may rotate the first metatarsal about the center point of rotation. This rotation results in plantar and/or dorsiflex dependent on the direction the carriage is translated.

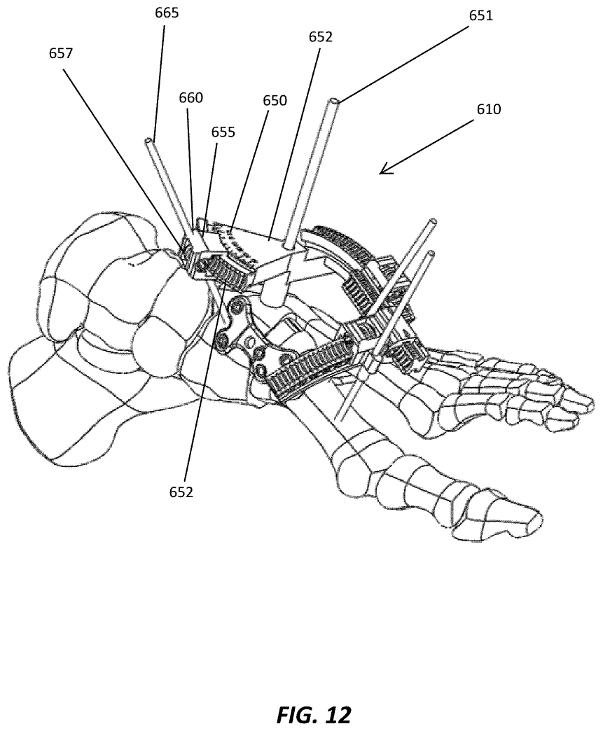

FIG. 12 depicts a bone displacement system 610, similar to bone displacement system 510, which further includes an intermetatarsal curved rail mechanism 650 that has a rail 652 and movable holding portion 655 with a worm gear 657. Holding portion 655 may be connected to a bone (e.g., first medial cuneiform 115) via a K-wire 651 extending through a hole therethrough. Holding portion 655 may include an opening 660 for receiving a K-wire 665 to connect holding portion 655 and mechanism 650 to a bone (e.g., first medial cuneiform 115). Such connection may allow movement of a first bone (e.g., first medial cuneiform 115) about a wire 651 extending through an anchoring portion 652 of intermetatarsal curved rail mechanism 650. The use of curved rail mechanism 650 may allow an alternative method of correcting bunion deformity, specifically an intermetatarsal angle. Movable holding portion 655 may move on rail 652 with a center of rotation about K-wire 651 where K-wire 651 may be placed in the center of a medial cuneiform (e.g., first medial cuneiform 115). Holding portion 655 may be connected to the medial cuneiform (e.g., medical cuneiform 115) via a K-wire 665 extending therethrough. Holding portion 655 may be translated along rail 652 to rotate holding portion 655 connected to the cuneiform about K-wire 651 placed in the center of the medial cuneiform.

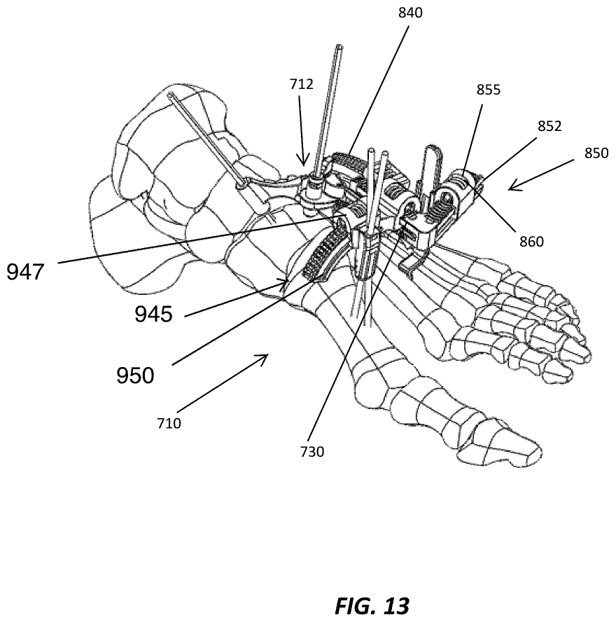

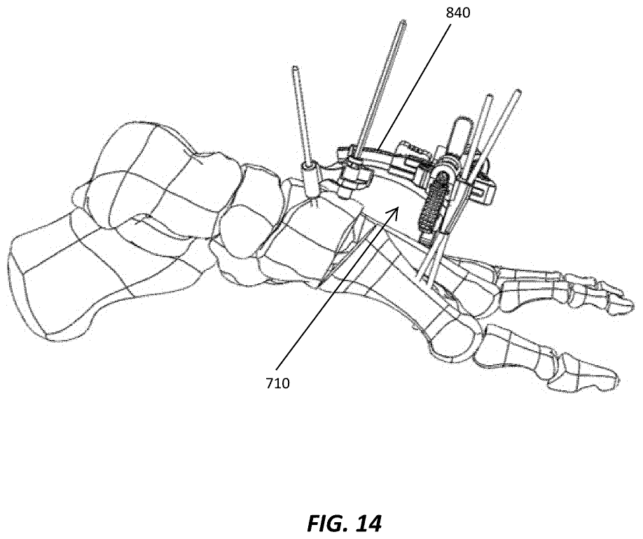

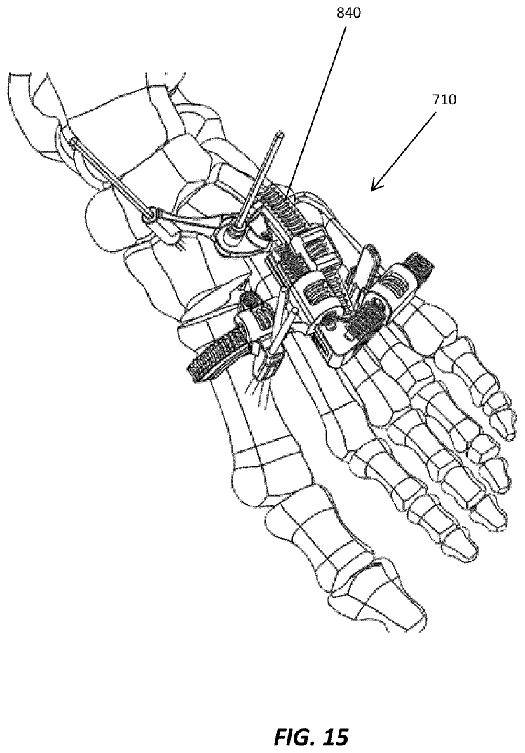

FIG. 13-15 depicts a bone displacement system or device 710 having features corresponding to those of bone displacement systems disclosed in co-owned patent application Ser. No. 17/238,920 (Attorney docket number 3768.099A) with the addition of a flexing mechanism 840 similar to flexing mechanism 340 described above. Flexing mechanism 840 may be connected to a proximal anchoring portion or cartridge engaging portion 712 and may be connected to a linear compression-distraction mechanism 730 similar to linear distraction mechanism 40 of the indicated patent application and mechanism 520 described above. Device 710 may include a lateral adjusting mechanism 850 having a rail 852, holding portion 855 and worm gear 860 in place of the ratchet mechanism disclosed in the co-owned patent application. A rotation mechanism 945 may include a holding portion 947 engageable with a rail 950 similar to mechanism 540 described above. As described

Further, device 710 may be connected to a paddle or cutting guide as described relative to bone displacement systems disclosed in co-owned patent application Ser. No. 17/238,920 (Attorney docket number 3768.099A). For example, cartridge engaging portion 712 may be connected to a paddle cartridge similar to paddle cartridge 50, and a saw guide similar to saw guide 250, as described and depicted in the indicated patent application. As described in the patent, the paddle cartridge may be utilized for initial positioning of the device relative to a joint location and the cut guide may allow for accurate bone resection.

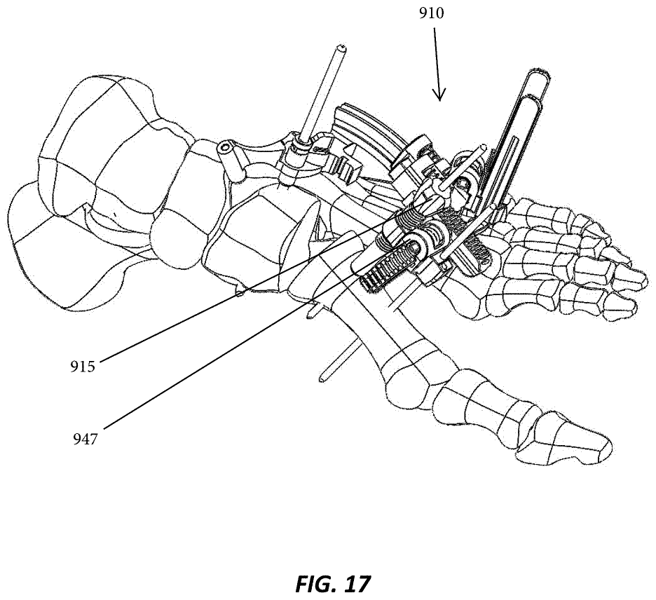

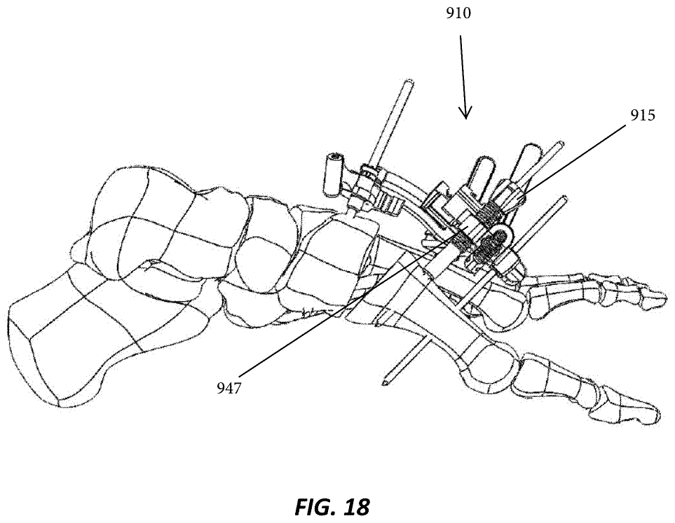

In another example depicted in FIGS. 17-18 a bone displacement system or device 910 may be identical to device 710 except that holding portion 947 may include a threaded push screw 915 cannulated to allow a k-wire to pass therethrough in place of an opening, such as opening 132 in the indicated patent, such that the k-wire may be inserted into a corresponding bone, such as a metatarsal. When translated, threaded push screw 947 may push or pull (i.e., with the assistance of soft tissue) a metatarsal plantar or dorsal relative to device 910 in an axial direction relative to an axis of push screw 947.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

We claim:

1. A bone displacement system comprising:
an anchoring portion having an aperture for receiving a wire to connect the anchoring portion to a proximal bone;
a flexing mechanism connected to said anchoring portion and having a rail and a holding portion movable on said rail, said rail being curved about a center of rotation located toward a bottom of said flexing mechanism and toward the proximal bone; and
a distal body connected to said holding portion, said distal body having an aperture for receiving a wire to connect said distal body to a distal bone, and said flexing mechanism configured to move said distal body relative to said anchoring portion by a movement of said holding portion on said rail and about the center of rotation to flex the first bone relative to the second bone.

2. The system of claim 1 further comprising a screw engaging the rail and the holding portion to allow movement of the holding portion and the distal bone relative to the lateral bone when a user drives said screw, the distal body is connected to the distal bone and the anchor is connected to the proximal bone.

3. The system of claim 1 further comprising a tool engaging portion connected to said anchoring portion and configured to connect a tool thereto.

4. The system of claim 3 wherein said tool comprises a paddle, the paddle releasably connected to the tool engaging portion and receivable between the proximal bone and the distal bone for locating the anchoring portion relative to the proximal bone and said distal body relative to the distal bone.

5. The system of claim 3 wherein said tool comprises a cut guide releasably connected to the tool engaging portion and having a first cut slot and a second cut slot for cutting the proximal bone and/or the distal bone.

6. The system of claim 1 further comprising a compression distraction fixture connected to said flex mechanism, wherein said compression distraction fixture comprises a rack mobile portion, a rack and a screw engageable by a user to cause movement of the rack mobile portion relative to the rack to cause a compression or a distraction of the proximal bone relative to the distal bone when the proximal bone is connected to the anchoring portion and the distal bone is connected to the distal body.

7. The system of claim 6 wherein the distal body comprises a trolley mobile portion moveably engaged with a rail and the rail has a rail longitudinal axis, the compression distraction fixture having an arm mobile portion engaged with a rack and the rack having a rack longitudinal axis, said rail longitudinal axis and said rack longitudinal axis being about perpendicular to each other.

8. A method for use in bone displacement, comprising:
inserting a first wire through an anchoring portion of a bone displacement mechanism into a first bone, the anchoring portion connected to a curved rail;
inserting a second wire through a distal body of the bone displacement mechanism distal to the anchoring portion into a second bone distal to the first bone, the distal body connected to a holding portion; and
moving the holding portion on the curved rail to move the distal body relative to the anchoring portion and about a center of rotation of the curved rail to flex the first bone relative to the second bone.

9. The method of claim 8 further comprising releasably connecting an alignment paddle to the bone displacement mechanism and inserting the paddle between a first bone and a second bone.

10. The method of claim 8 further comprising releasably connecting a bone cut guide to the bone displacement mechanism and cutting the first bone through a slot of the bone cut guide and cutting the second bone through a second slot of the bone cut guide.
